(19) United States
(12) Patent Application Publication
(10) Pub. No.: US 2019/0070212 A1
(43) Pub. Date: Mar. 7, 2019

(54) COMPOUNDS AND COMPOSITIONS FOR THE TREATMENT OF INFECTIONS

(71) Applicant: Spring Bank Pharmaceuticals, Inc., Milford, MA (US)

(72) Inventor: Radhakrishnan P. Iyer, Shrewsbury, MA (US)

(21) Appl. No.: 16/083,649

(22) PCT Filed: Mar. 10, 2017
(86) PCT No.: PCT/US2017/021773

§ 371 Date: Sep. 10, 2018

Related U.S. Application Data
(60) Provisional application No. 62/307,127, filed on Mar. 11, 2016.

(51) Int. Cl. A61K 31/7084; C07H 19/10; C07H 19/16

(52) U.S. Cl. CPC A61K 31/7084; C07H 19/16; C07H 19/10

(57) ABSTRACT

This invention relates to compounds and compositions for use in the treatment of microbial infections, e.g., HCV, HBV, and RSV.

### CLAIM OF PRIORITY

This application is a U.S. National Phase Application under 35 U.S.C. § 371 of International Application PCT/US2017/021773, filed Mar. 3, 2017, which claims priority to U.S. Application No. 62/307,127, filed Mar. 11, 2016, the contents of each of which are incorporated herein by reference in its entirety.

### FIELD OF INVENTION

This invention relates to compounds and compositions for use in the treatment of microbial infections, e.g., HCV, HBV, and RSV.

### BACKGROUND OF INVENTION

Infections caused by microbes including the Hepatitis C virus (HCV), Hepatitis B virus (HBV), and the respiratory syncytial virus (RSV) are a major public health concern. HCV is a leading cause of liver disease worldwide, with nearly 170 million people infected and about four million new infections each year (Shephard, C. W. et al, Lancet Infect Dis (2005) 5:558-567). About 80% of acutely infected HCV patients progress to chronic infection, 20% of whom develop cirrhosis within 25 years and have increased likelihood of liver failure and hepatocellular carcinoma (Kohli, A. et al, J Am Med Assoc (2014) 312:631-640). HCV is the leading cause of liver transplantation in the United States.

Similarly, chronic infection with hepatitis B virus (HBV) is responsible for approximately 1.2 million deaths per year worldwide due to HBV-associated liver diseases, such as hepatic cirrhosis, and hepatocellular carcinoma (HCC) (Levanchy, D. J Viral Hepatol (2004) 11:97-107). It is estimated that more than 2 billion people have serological evidence of previous or current HBV infection, and that over 350 million individuals are chronic carriers of HBV (Levanchy, D. J Viral Hepatol (2004) 11:97-107; Kwon H., Lok. A. S. Nat Rev Gastroenterol Hepatol (2011) 8:275-284). In the case of RSV, roughly 75,000 to 125,000 children are hospitalized due to RSV infection annually, and it has been attributed as the cause of death in 10,000 people over the age of 65 each year (Murata, Y. and Falsey, A. M. Antivir Ther (2007) 12:659-670). Nearly all children are infected with RSV by three years of age, with about half of these suffering only minor symptoms including cough, rhinorrhea, and low grade fever. Roughly 25-40% of children develop a lower respiratory illness indicative of bronchiolitis or pneumonia. Currently, only two drugs have been approved for the prophylaxis or treatment of RSV (Empey, K. M. et al, Clin Infect Dis (2010) 50:1258-1267).

Although treatments are available for some of these infections, namely HCV and HBV, the long term efficacy of most antimicrobial drugs is hampered by the rapid emergence of resistant mutants, high toxicity, unwarranted side effects, and the risk of relapse upon discontinuation of treatment. Accordingly, there is a critical need for a new generation of therapies to combat microbial infections.

### SUMMARY OF INVENTION

The present invention features compounds and compositions for the treatment of a subject infected with an infection (e.g., a viral infection, e.g., HCV, HBV, or RSV). In some embodiments, the compositions comprise a compound of Formula (III):

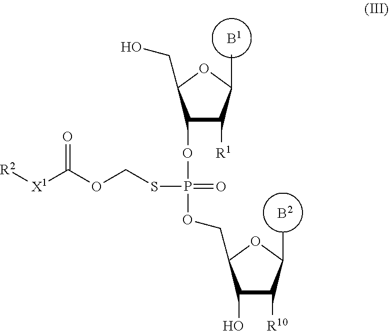

or a pharmaceutically acceptable salt thereof, wherein each of B¹ and B² is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); and each of R^{a} and R^{b} is independently H, C₁₋₆ alkyl, or aryl.

In some embodiments, each of B¹ and B² is independently adeninyl, thyminyl, uracilyl, or a modified nucleobase. In some embodiments, each of B¹ is independently thyminyl, uracilyl, or a modified nucleobase. In some embodiments, B¹ is uracilyl or a substituted uracilyl. In some embodiments, B² is adeninyl, cytosinyl, thyminyl, or a modified nucleobase. In some embodiments, B² is adeninyl or a substituted adeninyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, R¹⁰ is OH or OCH₃. In some embodiments, R¹⁰ is OH. In some embodiments, R¹⁰ is CH₃.

In some embodiments, the compound of Formula (III) is formulated as a composition. In some embodiments, the composition is formulated as a pharmaceutical composition.

In some embodiments, the composition is enantiomerically enriched for the Rp isomer over the Sp isomer. The amount of enantiomeric enrichment may be expressed as an ee ratio. In some embodiments, the composition comprises an ee ratio greater than about 51:49 of the Rp isomer to the Sp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Rp isomer to the Sp isomer. In other embodiments, the composition is enantiomerically enriched for the Sp isomer over the Rp isomer. In some embodiments, the composition comprises an ee ratio greater than about 51:49 of the Sp isomer to the Rp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Sp isomer to the Rp isomer.

In another aspect, the present invention features a compound of Formula (I):

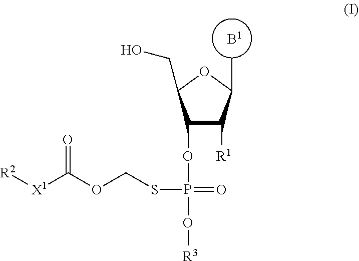

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl.

In some embodiments, B¹ is thyminyl, uracilyl, or a modified nucleobase. In some embodiments, B¹ is uracilyl or a substituted uracilyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, R³ is absent. In some embodiments, R³ is H.

In some embodiments, the compound of Formula (I) is a compound of Formula (I-a):

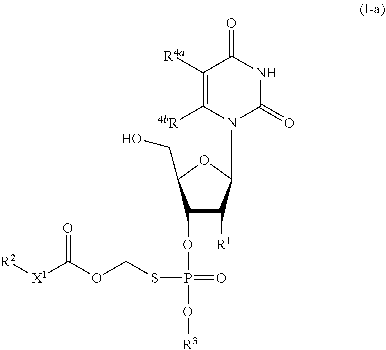

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, R³ is absent. In some embodiments, R³ is H.

In another aspect, the present invention features a compound of Formula (II):

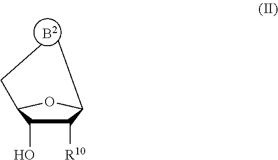

or a pharmaceutically acceptable salt thereof, wherein B² is adeninyl, cytosinyl, guanosinyl, thyminyl, uracilyl, or a modified nucleobase; and R¹⁰ is OH or O—(C₁₋₆ alkyl).

In some embodiments, B² is adeninyl, cytosinyl, thyminyl, or a modified nucleobase. In some embodiments, B² is adeninyl or a substituted adeninyl.

In some embodiments, R¹⁰ is OH or OCH₃. In some embodiments, R¹⁰ is OH. In some embodiments, R¹⁰ is OCH₃.

In some embodiments, the compound of Formula (II) is a compound of Formula (II-a):

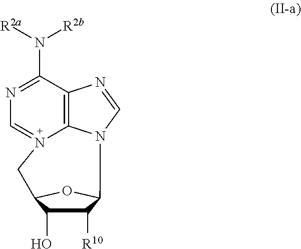

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

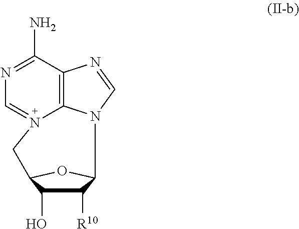

or a pharmaceutically acceptable salt thereof, wherein R¹⁰ is OH or O—(C₁₋₆ alkyl).

In some embodiments, the compound (e.g., a compound of Formula (I) or Formula (II) is a byproduct of degradation. In some embodiments, the compound (e.g., a compound of Formula (I) or Formula (II) is a byproduct of degradation of a compound of Formula (III). In some embodiments, the degradation is chemical degradation, physical degradation, thermal degradation, or pH-related degradation. In some embodiments, the degradation is thermal degradation. In some embodiments, the degradation is thermal degradation.

In another aspect, the present invention features a composition comprising a compound of Formula (III):

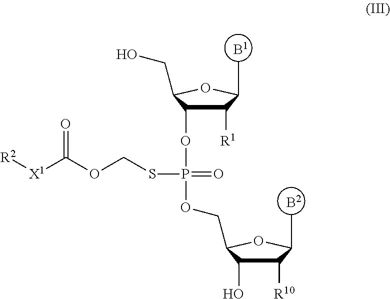

or a pharmaceutically acceptable salt thereof, wherein each of B¹ and B² is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); and each of R^{a} and R^{b} is independently H, C₁₋₆ alkyl, or aryl.

In some embodiments, the composition comprises a pharmaceutical composition.

In some embodiments, the composition is enantiomerically enriched for the Rp isomer over the Sp isomer (e.g., a compound of Formula (V-b) or (V-b)). The amount of enantiomeric enrichment may be expressed as an ee ratio. In some embodiments, the composition comprises an ee ratio greater than about 51:49 of the Rp isomer to the Sp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Rp isomer to the Sp isomer. In other embodiments, the composition is enantiomerically enriched for the Sp isomer over the Rp isomer. In some embodiments, the composition comprises an ee ratio greater than about 51:49 of the Sp isomer to the Rp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Sp isomer to the Rp isomer.

In some embodiments, the composition is substantially free of a compound of Formula (I):

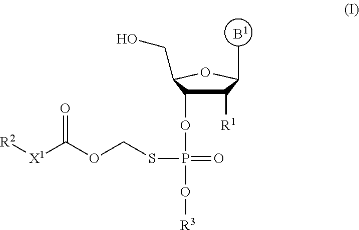

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl;
or a compound of Formula (II):

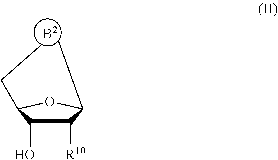

or a pharmaceutically acceptable salt thereof, wherein B² is adeninyl, cytosinyl, guanosinyl, thyminyl, uracilyl, or a modified nucleobase; R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, B¹ is thyminyl, uracilyl, or a modified nucleobase. In some embodiments, B¹ is uracilyl or a substituted uracilyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, R³ is absent. In some embodiments, R³ is H.

In some embodiments, the compound of Formula (I) is a compound of Formula (I-a):

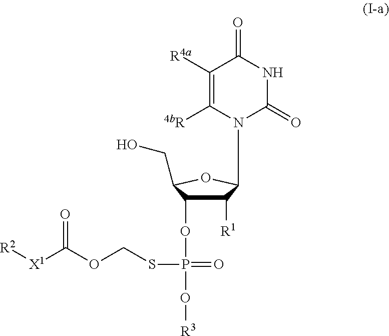

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, B² is adeninyl, cytosinyl, thyminyl, or a modified nucleobase. In some embodiments, B² is adeninyl or a substituted adeninyl.

In some embodiments, R¹⁰ is OH or OCH₃. In some embodiments, R¹⁰ is OH. In some embodiments, R¹⁰ is OCH₃.

In some embodiments, the compound of Formula (II) is a compound of Formula (II-a):

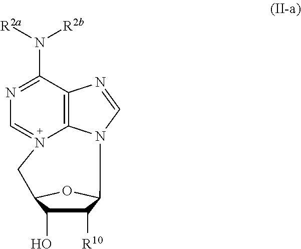

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

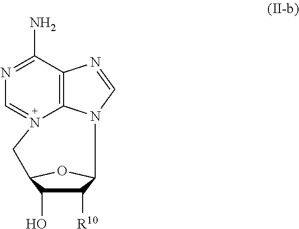

or a pharmaceutically acceptable salt thereof, wherein R¹⁰ is OH or O—(C₁₋₆ alkyl).

In another aspect, the present invention features a dosage form comprising a pharmaceutical composition, wherein the composition comprises a compound of Formula (III):

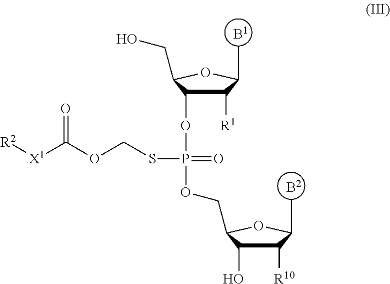

or a pharmaceutically acceptable salt thereof, wherein each of B¹ and B² is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); and each of R^{a} and R^{b} is independently H, C₁₋₆ alkyl, or aryl;
and the composition is substantially free of a compound of Formula (I):

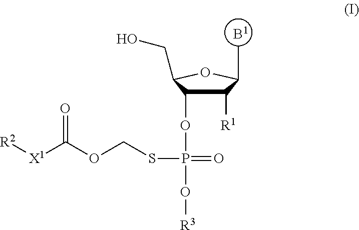

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl;
or a compound of Formula (II):

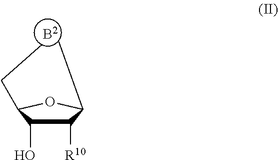

or a pharmaceutically acceptable salt thereof, wherein B² is adeninyl, cytosinyl, guanosinyl, thyminyl, uracilyl, or a modified nucleobase; and R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of B and B² is independently adeninyl, thyminyl, uracilyl, or a modified nucleobase. In some embodiments, each of B¹ is independently thyminyl, uracilyl, or a modified nucleobase. In some embodiments, B¹ is uracilyl or a substituted uracilyl. In some embodiments, B² is adeninyl, cytosinyl, thyminyl, or a modified nucleobase. In some embodiments, B² is adeninyl or a substituted adeninyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R³ is absent. In some embodiments, R³ is H.

In some embodiments, R¹⁰ is OH or OCH₃. In some embodiments, R¹⁰ of Formula (III) is OH. In some embodiments, R¹⁰ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, the compound of Formula (I) is a compound of Formula (I-a):

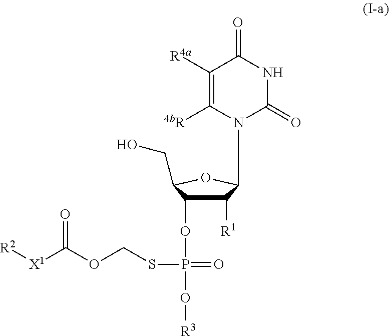

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, the compound of Formula (II) is a compound of Formula (II-a):

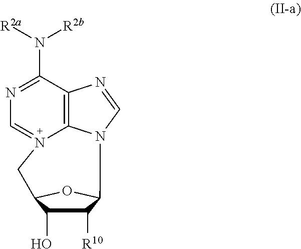

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

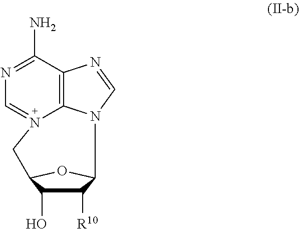

or a pharmaceutically acceptable salt thereof, wherein R¹⁰ is OH or O—(C₁₋₆ alkyl).

In some embodiments, the dosage form is administered orally, parenterally, or topically. In some embodiments, the dosage form is administered through inhalation. In some embodiments, the dosage form comprises a solid or liquid. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the composition further comprises an additive or preservative (e.g., PEG 400 or glycerin). In some embodiments, the composition further comprises an excipient (e.g., methylcellulose, talc, lactose, or starch).

### BRIEF DESCRIPTION OF THE DRAWINGS

FIGS. 1A-1F are depictions of predicted conformations of exemplary dinucleoside phosphorothioates based on quantum mechanical analyses (low energy states optimized at B3LYP 6-31G) as outlined in Example 1. FIGS. 1A-1B show the predicted conformations of the Rₚ-syn-isomer (FIG. 1A) and the Sₚ-syn-isomer (FIG. 1B) in the ground state. FIGS. 1C-1D show the predicted conformations of the Rₚ-syn-isomer (FIG. 1C) and the Sₚ-syn-isomer (FIG. 1D) in the transition state, while FIGS. 1E-1F illustrate the structures of two predicted degradation products, the cyclonucleoside (B, FIG. 1E) and the phosphorothioate diester (C, FIG. 1F).

### DETAILED DESCRIPTION OF THE INVENTION

The present invention relates to compounds and compositions for use in treating a subject infected with a microbial infection (e.g., HCV, HBV, or RSV) comprising a compound of Formula (III) or a pharmaceutically acceptable salt thereof.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, an amount of a compound, composition, or dosage form effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., a microbial infection, e.g., HCV, HBV, or RSV) beyond that expected in the absence of such treatment.

As used herein, the terms “prevent” or “preventing” as used in the context of a disorder or disease, refer to administration of an agent to a subject, e.g., the administration of a compound of the present invention (e.g., a compound of Formula (III) or a pharmaceutically acceptable salt thereof) such that the onset of at least one symptom of the disorder or disease is delayed as compared to what would be seen in the absence of administration of said agent.

As used herein, the term “subject” is intended to include human and non-human animals. Exemplary human subjects include a human patient having a disorder, e.g., a disorder described herein (e.g., a microbial infection, e.g., HCV, HBV, or RSV), or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of a composition comprising a compound of Formula (III) or a pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 100 to 1000 mg/day, excluding the lower endpoint, would cover an amount greater than 100 that is less than or equal to 1000 mg/day.

“Co-administration”, “co-administering” or “co-providing”, as used herein in the context of the administration of therapies, refers to administration at the same time, administration of one therapy before (e.g., immediately before, less than about 5, about 10, about 15, about 30, about 45, about 60 minutes, about 1, about 2, about 3, about 4, about 6, about 8, about 10, about 12, about 16, about 20, about 24, about 48, about 72 or more hours before) administration of a secondary therapy.

“Course of therapy”, as referred to herein, comprises one or more separate administrations of a therapeutic agent (e.g., a composition comprising a compound of Formula (III) or a pharmaceutically acceptable salt thereof). A course of therapy can comprise one or more cycles of a therapeutic agent.

A “cycle”, as used herein in the context of a cycle of administration of a drug, refers to a period of time for which a drug is administered to a patient. For example, if a drug is administered for a cycle of 4 weeks days, the periodic administration, e.g., daily or twice daily, is given for 4 weeks. A drug can be administered for more than one cycle. In some embodiments, the first and second or subsequent cycles are the same in terms of one or both of duration and periodic administration. In embodiments, a first and second or subsequent cycle differs in terms of one or both of duration and periodic administration. Rest periods may be interposed between cycles. A rest cycle may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

### Chemical Definitions

Definitions of specific functional groups and chemical terms are described in more detail below. The chemical elements are identified in accordance with the Periodic Table of the Elements, CAS version, Handbook of Chemistry and Physics, 75^{t} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{h} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

At various places in the present specification, substituents of compounds of the invention are disclosed in groups or in ranges. It is specifically intended that the invention include each and every individual subcombination of the members of such groups and ranges. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. For compounds of the invention in which a variable appears more than once, each variable can be a different moiety selected from the Markush group defining the variable. For example, where a structure is described having two R groups that are simultaneously present on the same compound, the two R groups can represent different moieties selected from the Markush group defined for R.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, “alkyl,” by itself or as part of another substituent, means, unless otherwise stated, a straight or branched chain, and can have a number of carbon atoms as optionally designated (i.e., C₁-C₆ refers to an alkyl chain comprising one to six carbons).

Examples of saturated hydrocarbon groups include, but are not limited to, groups such as methyl, ethyl, n-propyl, isopropyl, n-butyl, t-butyl, isobutyl, sec-butyl, n-pentyl, isopentyl, homologs and isomers of, for example, n-pentyl, n-hexyl, and the like.

As used herein, “alkenyl” can be a straight or branched hydrocarbon chain, containing at least one double bond, and having from two to six carbon atoms (i.e. C₂-C₆ alkenyl). Examples of alkenyl groups, include, but are not limited to, groups such as ethenyl (i.e., vinyl), prop-1-enyl (i.e., allyl), but-1-enyl, pent-1-enyl, penta-1,4-dienyl, and the like.

As used herein, “alkynyl” can be a straight or branched hydrocarbon chain, containing at least one triple bond, having from two to six carbon atoms (i.e. C₂-C₆ alkynyl). Examples of alkynyl groups, include, but are not limited to, groups such as ethynyl, propynyl, butynyl, pentynyl, hexynyl, and the like.

As used herein, “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more cycloalkyl or heterocyclyl groups wherein the radical or point of attachment is on the aryl ring, and in such instances, the number of carbon atoms continue to designate the number of carbon atoms in the aryl ring system. Aryl groups include, but are not limited to, phenyl, naphthyl, indenyl, and tetrahydronaphthyl.

As used herein, “arylalkyl” refers to an (aryl)alkyl-radical wherein aryl and alkyl moieties are as disclosed herein.

As used herein, “heteroalkyl” refers to an alkyl that has one or more skeletal chain atoms selected from an atom other than carbon, e.g., oxygen, nitrogen, sulfur, phosphorus or combinations thereof. A numerical range may be given, e.g. C₁-C₆ heteroalkyl which refers to the number of carbons in the chain, which in this example includes 1 to 6 carbon atoms. For example, a —CH₂OCH₂CH₃ radical is referred to as a “C₃” heteroalkyl. Connection to the rest of the molecule may be through either a heteroatom or a carbon in the heteroalkyl chain.

As used herein, “hydroxy” or “hydroxyl” refers to a —OH radical.

As used herein, the term “nucleobase” refers to adeninyl, cytosinyl, guanosinyl, thyminyl, uracilyl, or a modified analog thereof. In some embodiments, a modified nucleobase comprises a chemical analog of a naturally occurring nucleobase (e.g., adeninyl, cytosinyl, guanosinyl, thyminyl, uracilyl), or variant thereof. Modified nucleobases may comprise chemical substitutions or substituents, e.g., the presence or absence of a heteroatom in a ring system, or addition of an alkyl, alkenyl, alkynyl, heteroalkyl, heteralkenyl, heteroalkynyl, aryl, heteroaryl, cyclyl, heterocyclyl, halo, or cyano group, or other permissible substituent. In some embodiments, each of the foregoing substituents may be further substituted.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

### Compounds and Therapeutic Agents

The present invention features compounds and compositions for the treatment of a subject infected with an infection (e.g., a viral infection, e.g., HCV, HBV, or RSV). In some embodiments, the compositions comprise a compound of Formula (III):

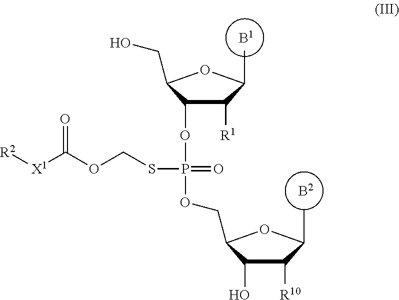

or a pharmaceutically acceptable salt thereof, wherein each of B¹ and B² is adeninyl, cytosinyl, guanosinyl, thyminyl, uracilyl, or a modified nucleobase; X¹ is absent, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); and each of R^{a} and R^{b} is independently H, C₁₋₆ alkyl, or aryl.

In some embodiments, each of B¹ and B² is independently adeninyl, thyminyl, uracilyl, or a modified nucleobase. In some embodiments, each of B¹ is independently thyminyl, uracilyl, or a modified nucleobase. In some embodiments, B¹ is uracilyl or a substituted uracilyl. In some embodiments, B² is adeninyl, cytosinyl, thyminyl, or a modified nucleobase. In some embodiments, B² is adeninyl or a substituted adeninyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b} (e.g., C(O)OC(CH₃)₃).

In some embodiments, R¹⁰ is OH or OCH₃. In some embodiments, R¹⁰ is OH. In some embodiments, R¹⁰ is CH₃.

In some embodiments, the compound of Formula (III) is formulated as a composition. In some embodiments, the composition is formulated as a pharmaceutical composition.

In some embodiments, the compound of Formula (III) or a pharmaceutically acceptable salt thereof is a prodrug, and the active agent is the compound of Formula (V), which may be described by any one of Formula (V-a), Formula (V-b), and Formula (V-c), or pharmaceutically acceptable salt or a combination thereof:

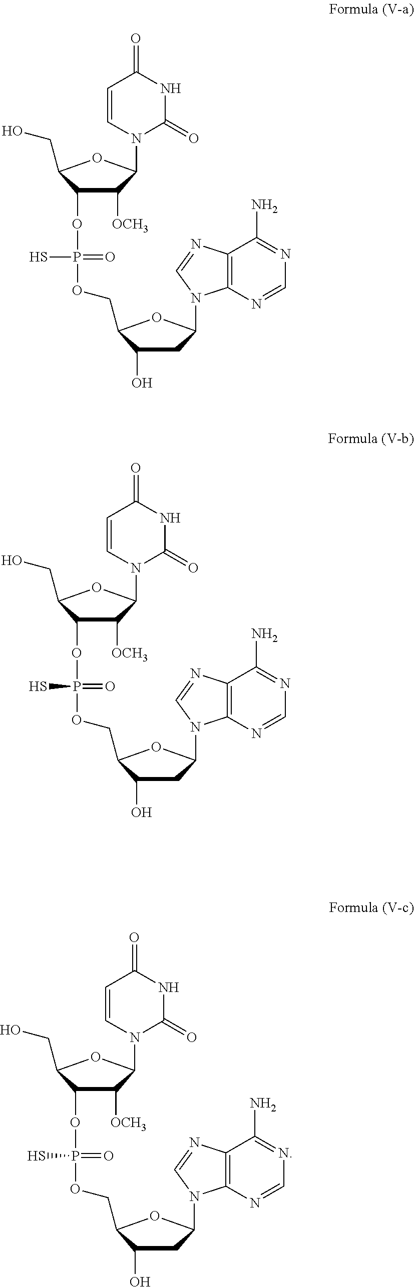

A compound of Formula (III) or a pharmaceutically salt thereof, and the active agent Formula (V) (e.g. a compound of Formula (V-a), (V-b), (V-c), a pharmaceutically acceptable salt or combination thereof) are small molecule nucleic acid hybrid (dinucleotide) compounds that combine both antiviral and immune modulating activities. The latter activity mediates controlled apoptosis of virus-infected hepatocytes via stimulation of the innate immune response, similar to what is also achieved by IFN-α therapy in HBV-infected patients.

Without wishing to be bound by theory, the mechanism of action of a compound of Formulas (III) or (V) may be dissected into two components. The first component entails the host immune stimulating activity of Formulas (III) or (V) which induces endogenous IFNs via the activation of viral sensor proteins, e.g., retinoic acid-inducible gene 1 (RIG-I) and nucleotide-binding oligomerization domain-containing protein 2 (NOD2) (Takeuchi, O. and Akira S. Cell (2010) 140:805-820; Sato, S. et al. Immunity (2015) 42:123-132; Sabbah, A. et al. Nat Immunol (2009) 10:1073-1080). Activation may occur by binding of Formula (I) to the RIG-I/NOD2 proteins at their nucleotide binding domain. The RIG-I and NOD2 proteins are located in the cytosol of cells, including hepatocytes, and usually recognize signature patterns of foreign nucleic acids such as the pathogen associated molecular pattern (PAMP). Once PAMP within viral RNA or DNA is recognized, RIG-I and NOD2 may become activated and trigger the IFN signaling cascade that then results in IFN and interferon-stimulated gene (ISG) production and induction of an antiviral state in cells. In the case of HDV, the PAMP is believed to be any of the genomic or other RNA species which has secondary structure.

The second component of the mechanism of action of Formulas (III) or (V) involves its direct antiviral activity, which inhibits the synthesis of viral nucleic acids by steric blockage of the viral polymerase. The block may be achieved by interaction Formula (V) with RIG-I and NOD2 as described above that then in turn may prevent the polymerase enzyme from engaging with the viral nucleic acid template for replication (i.e., HBV pre-genomic RNA). The cytotoxic potential of Formula (V) has been initially evaluated using a panel of cell lines. Formula (V) demonstrated an excellent safety profile, with a 50% cytotoxic concentration (CC50) of greater than 1000 μM (Coughlin, J. E. et al. Bioorg Med Chem Lett (2010) 20:1783-1786). Formula (V) has been further evaluated for anti-HBV activity in a cell-based assay against wild-type HBV and against lamivudine- (3TC) and adefovir- (ADV) resistant mutant HBV. Formula (V) was found to have antiviral activity against wild-type HBV, with a potency that was in the range of ADV (but less than that of 3TC).

It is well established that the prodrug of Formula (III) has been shown to be converted to the active drug Formula (V) (e.g., the Rp- and Sp-Formula (V) isomers, Formula (V-b) and Formula (V-c)) upon administration. In some embodiments, the composition is enantiomerically enriched for the Rp isomer over the Sp isomer. The amount of enantiomeric enrichment may be expressed as an ee ratio. In some embodiments, the composition comprises an ee ratio greater than about 51:49 of the Rp isomer to the Sp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Rp isomer to the Sp isomer. In other embodiments, the composition is enantiomerically enriched for the Sp isomer over the Rp isomer. In some embodiments, the composition comprises an ee ratio greater than about 51:49 of the Sp isomer to the Rp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Sp isomer to the Rp isomer.

In another aspect, the present invention features a compound of Formula (I):

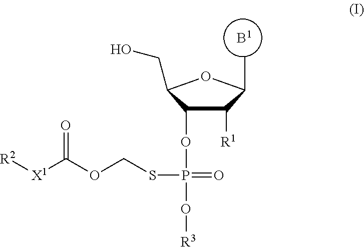

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl.

In some embodiments, B¹ is thyminyl, uracilyl, or a modified nucleobase. In some embodiments, B¹ is uracilyl or a substituted uracilyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, R³ is absent. In some embodiments, R³ is H.

In some embodiments, the compound of Formula (I) is a compound of Formula (I-a):

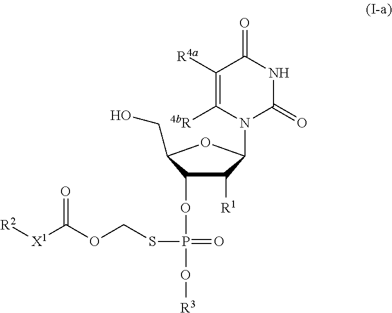

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, R³ is absent. In some embodiments, R³ is H.

In another aspect, the present invention features a compound of Formula (II):

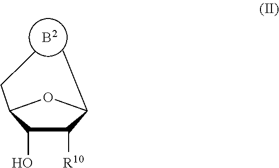

or a pharmaceutically acceptable salt thereof, wherein B² is adeninyl, cytosinyl, guanosinyl, thyminyl, uracilyl, or a modified nucleobase; and R¹⁰ is OH or O—(C₁₋₆ alkyl).

In some embodiments, B² is adeninyl, cytosinyl, thyminyl, or a modified nucleobase. In some embodiments, B² is adeninyl or a substituted adeninyl.

In some embodiments, R¹⁰ is OH or OCH₃. In some embodiments, R¹ is OH. In some embodiments, R¹⁰ is OCH₃.

In some embodiments, the compound of Formula (II) is a compound of Formula (II-a):

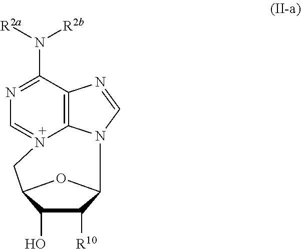

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

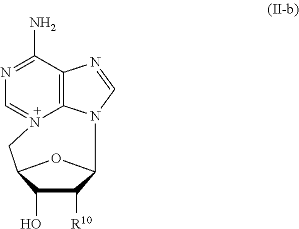

or a pharmaceutically acceptable salt thereof, wherein R¹⁰ is OH or O—(C₁₋₆ alkyl).

In some embodiments, the compound (e.g., a compound of Formula (I) or Formula (II) is a byproduct of degradation. In some embodiments, the compound (e.g., a compound of Formula (I) or Formula (II) is a byproduct of degradation of a compound of Formula (III). In some embodiments, the degradation is chemical degradation, physical degradation, thermal degradation, or pH-related degradation. In some embodiments, the degradation is thermal degradation. In some embodiments, the degradation is thermal degradation.

In another aspect, the present invention features a composition comprising a compound of Formula (III):

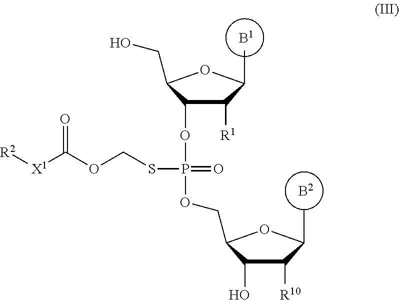

or a pharmaceutically acceptable salt thereof, wherein each of B¹ and B² is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); and each of R^{a} and R^{b} is independently H, C₁₋₆ alkyl, or aryl.

In some embodiments, the composition comprises a pharmaceutical composition.

In some embodiments, the composition is enantiomerically enriched for the Rp isomer over the Sp isomer. The amount of enantiomeric enrichment may be expressed as an ee ratio. In some embodiments, the composition comprises an ee ratio greater than about 51:49 of the Rp isomer to the Sp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Rp isomer to the Sp isomer. In other embodiments, the composition is enantiomerically enriched for the Sp isomer over the Rp isomer. In some embodiments, the composition comprises an ee ratio greater than about 51:49 of the Sp isomer to the Rp isomer. In some embodiments, the composition comprises an ee ratio greater than about 55:45, about 60:40, about 65:35, about 70:30, about 75:25, about 80:20, about 85:15, about 90:10, about 95:5, or about 99:1 of the Sp isomer to the Rp isomer.

In some embodiments, the composition is substantially free of a compound of Formula (I):

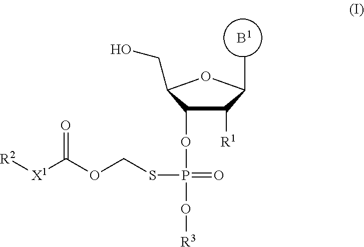

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl; or a compound of Formula (II):

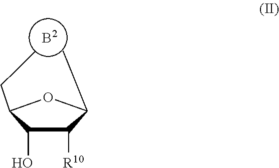

or a pharmaceutically acceptable salt thereof, wherein B² is adeninyl, cytosinyl, guanosinyl, thyminyl, uracilyl, or a modified nucleobase; R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, B¹ is thyminyl, uracilyl, or a modified nucleobase. In some embodiments, B¹ is uracilyl or a substituted uracilyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, R³ is absent. In some embodiments, R³ is H.

In some embodiments, the compound of Formula (I) is a compound of Formula (I-a):

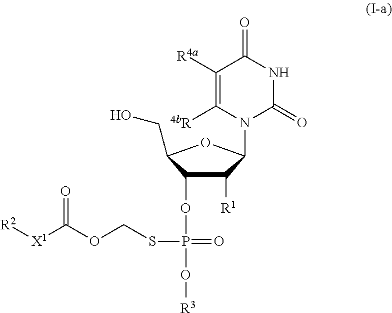

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, B² is adeninyl, cytosinyl, thyminyl, or a modified nucleobase. In some embodiments, B² is adeninyl or a substituted adeninyl.

In some embodiments, R¹⁰ is OH or OCH₃. In some embodiments, R¹⁰ is OH. In some embodiments, R¹⁰ is OCH₃.

In some embodiments, the compound of Formula (II) is a compound of Formula (II-a):

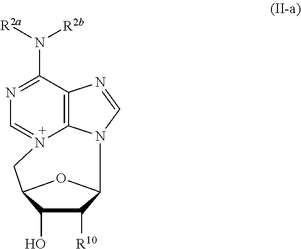

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

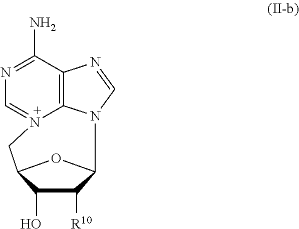

or a pharmaceutically acceptable salt thereof, wherein R¹⁰ is OH or O—(C₁₋₆ alkyl).

In another aspect, the present invention features a dosage form comprising a pharmaceutical composition, wherein the composition comprises a compound of Formula (III):

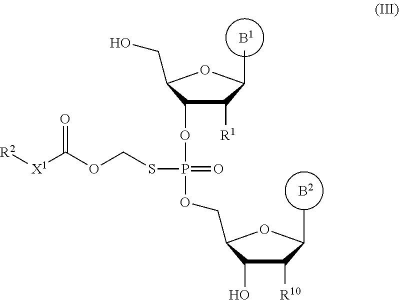

or a pharmaceutically acceptable salt thereof, wherein each of B¹ and B² is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R⁴ is C₁₋₆ alkyl, C₁₋₆ heteroalkyl, aryl, C(O)R^{a}, or C(O)OR^{b}; R¹⁰ is H or O—(C₁₋₆ alkyl); each of R^{a} and R^{b} is independently H, C₁₋₆ alkyl, or aryl; and the composition is substantially free of a compound of Formula (I):

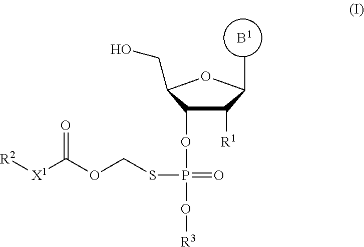

or a pharmaceutically acceptable salt thereof, wherein B¹ is adeninyl, cytosinyl, guanosinyl, thyminyl, uracilyl, or a modified nucleobase; X¹ is absent, O, or C₁₋₆ alkyl; R¹ is OH or O—(C₁₋₆ alkyl); R² is C₁₋₆ alkyl, C₁₋₆ heteroalkyl, or arylalkyl, wherein each alkyl, heteroalkyl, or arylalkyl is optionally substituted with 1-5 R⁴; R³ is absent or H; R⁴ is C₁₋₆ alkyl, C₁₋₆ heteroalkyl, aryl, C(O)R^{a}, or C(O)OR^{b}; and each of R^{a} and R^{b} is independently H, C₁₋₆ alkyl, or aryl;
or a compound of Formula (II):

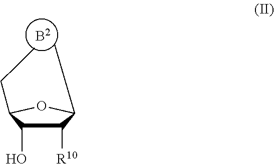

or a pharmaceutically acceptable salt thereof, wherein B is adeninyl, cytosinyl, guanosinyl, thyminyl, uracilyl, or a modified nucleobase; and R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of B and B² is independently adeninyl, thyminyl, uracilyl, or a modified nucleobase. In some embodiments, each of B¹ is independently thyminyl, uracilyl, or a modified nucleobase. In some embodiments, B¹ is uracilyl or a substituted uracilyl. In some embodiments, B² is adeninyl, cytosinyl, thyminyl, or a modified nucleobase. In some embodiments, B² is adeninyl or a substituted adeninyl.

In some embodiments, X¹ is absent, O, or CH₂. In some embodiments, X¹ is absent. In some embodiments, X¹ is O. In some embodiments, X¹ is CH₃.

In some embodiments, R¹ is OH or OCH₃. In some embodiments, R¹ is OH. In some embodiments, R¹ is CH₃.

In some embodiments, R³ is absent. In some embodiments, R³ is H.

In some embodiments, R¹⁰ is OH or OCH₃. In some embodiments, R¹⁰ of Formula (III) is OH. In some embodiments, R¹⁰ is CH₃.

In some embodiments, R² is C₁₋₆ alkyl (e.g., C(CH₃)₃, CH(CH₃)₂). In some embodiments, R² is C₁₋₆ heteroalkyl (e.g., CH(NH(R⁴))CH₂R⁴, CH₂CH₂OC(CH₃)₃). In some embodiments, R² is arylalkyl.

In some embodiments, R⁴ is aryl (e.g., phenyl), C(O)R^{a} (e.g., C(O)CH₃) or C(O)OR^{b}(e.g., C(O)OC(CH₃)₃).

In some embodiments, the compound of Formula (I) is a compound of Formula (I-a):

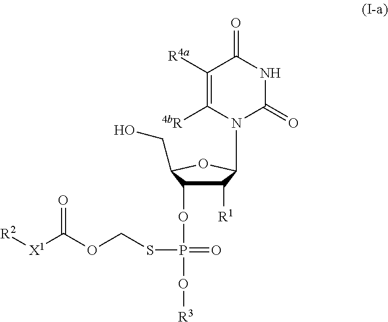

or a pharmaceutically acceptable salt thereof, wherein each of R¹, R², and R³ are as defined as for Formula (I), and each of R^{4a} and R^{4b} is independently H or C₁₋₆ alkyl, C₁₋₆ alkenyl, or C₁₋₆ alkynyl.

In some embodiments, each of R^{4a} and R^{4b} is independently H. In some embodiments, one of R^{4a} and R^{4b} is independently H and the other of R^{4a} and R^{4b} is C₁₋₆ alkyl, C₁₋₆ alkenyl, or C₁₋₆ alkynyl. In some embodiments, one of R^{4a} and R^{4b} is independently H and the other of R^{4a} and R^{4b} is C₁₋₆ alkynyl (e.g., pentynyl).

In some embodiments, the compound of Formula (II) is a compound of Formula (II-a):

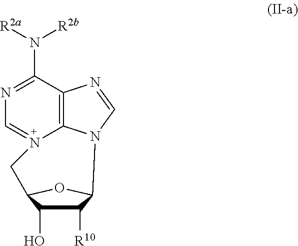

or a pharmaceutically acceptable salt thereof, wherein R¹⁰ is OH or O—(C₁₋₆ alkyl); each of R^{2a} and R^{2b} is independently H, C₁₋₆ alkyl, halogen, C(O)R^{c}, or C(O)OR^{d}; and each of R^{c} and R^{d} is independently H, C₁₋₆ alkyl, or aryl.

In some embodiments, each of R^{2a} and R^{2b} is independently H or C(O)OR^{b} (e.g., C(O)OCH(CH₃)₂). In some embodiments, each of R^{2a} and R^{2b} is independently H. In some embodiments, one of R^{2a} and R^{2b} is H and the other of R^{2a} and R^{2b} is C(O)R^{c} or C(O)OR^{d} (e.g., C(O)-aryl or C(O)O—C₁₋₆ alkyl). In some embodiments, one of R^{2a} and R^{2b} is H and the other of R^{2a} and R^{2b} is C(O)R^{c} or C(O)OR^{d} (e.g., C(O)-phenyl or C(O)O—CH(CH₃)₂).

In some embodiments, the compound of Formula (II) is a compound of Formula (II-b):

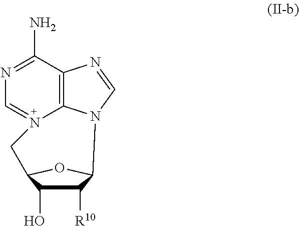

or a pharmaceutically acceptable salt thereof, wherein R¹⁰ is OH or O—(C₁₋₆ alkyl).

The compounds provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

### Additional Agents

The present invention features compounds and compositions for treating a subject infected with an infection, e.g., a microbial infection (e.g., an HCV, HBV, or RSV infection). In some embodiments of the present invention, the subject is further administered an additional agent or treatment in conjunction with a compound of Formula (III). In some embodiments, the additional agent may be an agent for treating a microbial infection, e.g., an HCV, HBV, or RSV infection. In some embodiments, the additional agent is an antiviral agent or an anticancer agent. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, simeprevir, or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

In some embodiments, the nucleoside analog comprises lamivudine, adefovir dipivoxil, entecavir, telbivudine, clevudine, ribavarin, tenofovir, tenofovir dipivoxil, tenofovir alafenamide, besifovir, or AGX-1009. In some embodiments, the antiviral agent is entecavir. In some embodiments, the antiviral compound comprises NOV-225, BAM 205, Myrcludex B, ARC-520, BAY 41-4109, REP 9AC, Alinia (nitazoxanide), Dd-RNAi, NVR-121 (NVR 3-778), BSBI-25, NVP-018, TKM-HBV, or ALN-HBV. In some embodiments, the non-interferon immune enhancer comprises zadaxin (thymosin alpha-1), GS-4774, CYT107 (interleukin-7), Dv-601, HBV core antigen vaccine, or GS-9620. In some embodiments, the antiviral agent is a capsid inhibitor, an entry inhibitor, a secretion inhibitor, a microRNA, an antisense RNA agent, an RNAi agent, or other agent designed to inhibit viral RNA. In some embodiments, the anticancer agent is selected from methotrexate, 5-fluorouracil, doxorubicin, vincristine, bleomycin, vinblastine, dacarbazine, toposide, cisplatin, epirubicin, and sorafenib tosylate.

In some embodiments, the combination of a compound of Formula (III) and the additional agent has a synergistic or additive effect. In some embodiments, the term “additive” refers to an outcome wherein when two agents are used in combination, the combination of the agents acts in a manner equal to but not greater than the sum of the individual anti-microbial activities of each agent.

In some embodiments, the terms “synergy” or “synergistic” refer to an outcome wherein when two agents are used in combination, the combination of the agents acts so as to require a lower concentration of each individual agent than the concentration required to be efficacious in the absence of the other agent. In some embodiments, a synergistic effect results in a reduced in a reduced minimum inhibitory concentration of one or both agents, such that the effect is greater than the sum of the effects. A synergistic effect is greater than an additive effect. In some embodiments, the agents in the composition herein may exhibit a synergistic effect, wherein the anti-HCV activity at a particular concentration is greater than at least about 1.25, 1.5, 1.75, 2, 2.5, 3, 4, 5, 10, 12, 15, 20, 25, 50, or 100 times the anti-microbial activity of either agent alone.

### Pharmaceutical Compositions

The present invention features methods for treating a subject infected with a microbial infection (e.g., an HCV, HBV, or RSV infection) with a composition comprising a compound of Formula (III), or a pharmaceutically acceptable salt thereof. The methods described herein may comprise administration of a composition of a compound of Formula (III) or a pharmaceutically acceptable salt thereof, and this composition may be combined with one or more pharmaceutically acceptable diluents, excipients, or carriers. The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting (e.g., a compound of Formula (III)). Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound of Formula (III)) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylacticaly effective amount of a composition comprising a compound of Formula (III), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts without limitation include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the compound of the present invention (e.g., a compound of Formula (III)). These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form. The pharmaceutical composition may be in unit dosage forms suitable for single administration of precise dosages. Pharmaceutical compositions may comprise, in addition to the compounds described herein (e.g., a compound of Formula (III) or a pharmaceutically acceptable salt thereof), a pharmaceutically acceptable carrier, and may optionally further comprise one or more pharmaceutically acceptable additives, preservatives, or excipients, such as, for example, stabilizers (e.g., a binder, e.g., polymer, e.g., a precipitation inhibitor, diluents, binders, and lubricants. In some embodiments, the composition further comprises an additive or preservative (e.g., PEG 400 or glycerin). In some embodiments, the composition further comprises an excipient (e.g., methylcellulose, talc, lactose, or starch).

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising compounds of the present invention (e.g., a compound of Formula (III)) or pharmaceutically acceptable salts thereof.

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of compounds of the present invention (e.g., a composition comprising a compound of Formula (III)), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present invention is accomplished by dissolving or suspending compound in an oil vehicle.

In some embodiments, it may be advantageous to administer the compounds (e.g., a compound of Formula (III)) and compositions of the present invention in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time.

In some embodiments, the compositions described herein comprising a compound of Formula (III) is administered orally. In some embodiments, the compositions described herein comprising a compound of Formula (III) is administered parenterally. In some embodiments, the compositions described herein comprising a compound of Formula (III) is administered topically. In exemplary embodiments of the invention, the compositions described herein comprising a compound of Formula (III) is administered intravenously. In some embodiments, the compositions described herein comprising a compound of Formula (III) is administered intranasally (e.g., via inhalation).

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

### Dosages

The compositions of the present invention are formulated into acceptable dosage forms by conventional methods known to those of skill in the art. Actual dosage levels of the active ingredients in the compositions of the present invention (e.g., a compound of Formula (III)) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally) to a subject afflicted with the disorders described herein (e.g., HCV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of compounds of Formula (III) or a pharmaceutically acceptable salt thereof for the treatment of a microbial infection (e.g., a viral infection). Accordingly, a patient and/or subject can be selected for treatment using a compound of Formula (III) or a pharmaceutically acceptable salt thereof by first evaluating the patient and/or subject to determine whether the subject is infected with a microbial infection (e.g., viral infection). In some embodiments, the patient and/or subject is selected for treatment using a compound of Formula (III) or a pharmaceutically acceptable salt thereof through determination of the serotypic and genotypic classification of the virus (e.g., the virus infecting the patient and/or subject). A subject can be evaluated as infected with a microbial infection (e.g., a viral infection) using methods known in the art. The subject can also be monitored, for example, subsequent to administration of a composition described herein (e.g., a compound of Formula (III)) or a pharmaceutically acceptable salt thereof.

In some embodiments, the subject is a mammal. In some embodiments, the subject is a human. In some embodiments, the subject is an adult (e.g., over about 18 years of age, over about 35 years of age, over about 65 years of age, over about 80 years of age). In some embodiments, the subject is a child (e.g, under about 5 years of age, under about 4 years or age, under about 3 years of age, under about 2 years of age, under about 1 year of age).

In some embodiments, the subject has been diagnosed with an HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11.

In some embodiments, the subject has been diagnosed with an HBV infection. In some embodiments, the subject is diagnosed with chronic HBV. In some embodiments, the genotype of the HBV infection is known. In some embodiments, the subject has received previous treatment for HBV.

In some embodiments, the subject has been diagnosed with an RSV infection. In some embodiments, the subject is suffering from a severe RSV infection. In some embodiments, the genotype, serotype, subtype, or antigenic grouping of the RSV infection is known (e.g., RSV-A or RSV-B). In some embodiments, the subject is immunocompromised (e.g., a subject that may have a weakened immune system relative to a reference standard, or may be suffering from an immune disease or condition). In some embodiments, the subject is suffering from bronchiolitis, pneumonia, or other respiratory illness or condition. In some embodiments, the subject is further diagnosed with a cardiac illness or condition. In some embodiments, the subject is further diagnosed with a bacterial infection.

In some embodiments, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, the subject is treatment naïve. In some embodiments, the subject has previously been treated for an infection (e.g., a microbial infection, e.g., an HCV, HBV, or RSV infection) In some embodiments, the subject has been treated with an anti-microbial agent other than a compound of Formula (III) or a pharmaceutically acceptable salt thereof. In some embodiments, the subject has been treated with an interferon, a nucleoside analog, a non-nucleoside antiviral, a non-interferon immune enhancer, or a direct-acting antiviral. In some embodiments, the subject has been treated with an interferon, e.g., peg-interferon alfa (e.g., peg-interferon alfa-2a or peg-interferon alfa-2b). In some embodiments, the subject has been treated with ribavirin. In some embodiments, the subject has been treated with a viral protease inhibitor, e.g., an inhibitor of the NS3/4A protease, e.g., telaprevir, ciluprevir, boceprevir, paritaprevir, simeprevir or asunaprevir. In some embodiments, the subject has been treated with a NS5A inhibitor, e.g., ledipasvir, ombitasvir, dasabuvir, or daclatsavir. In some embodiments, the subject has been treated with a NS5B inhibitor, e.g., sofosbuvir.

In some embodiments, the subject has been further diagnosed with an HIV infection. In some embodiments, the strain of HIV infection is known. In some embodiments, the subject is infected with HIV-1 or HIV-2 (e.g., strain 1 or strain 2).

### EXAMPLES

### Example 1. Thermal Degradation Studies of Exemplary Dinucleotide Phosphorothioate Analogs

Exemplary dinucleotide phosphorothioates of the invention were assembled using the appropriate protected nucleosides via standard phosphoramidite chemistry, followed by chemoselective S-alkylation with iodoalkyl derivatives. Exemplary synthetic protocols are detailed in U.S. Pat. Nos. 6,881,831 and 8,076,303, each of which is incorporated herein by reference in its entirety. For thermal degradation studies, the S-alkylated derivatives were subjected to heating at 80° C. for 24 hours. The resulting products and the extent of degradation were identified by LC-MS and are outlined in Table 1. In selected instances, the products were purified by chromatography and additionally characterized by NMR spectroscopy. Based on the degradation products, fragmentation was determined to proceed as depicted in Scheme 1. Further structure confirmation was achieved by total synthesis. Molecular dynamics and quantum mechanical studies were conducted to further elucidate the mechanism of product formation and to provide a rational basis for structure and stereochemical related effects on the fragmentation reaction.

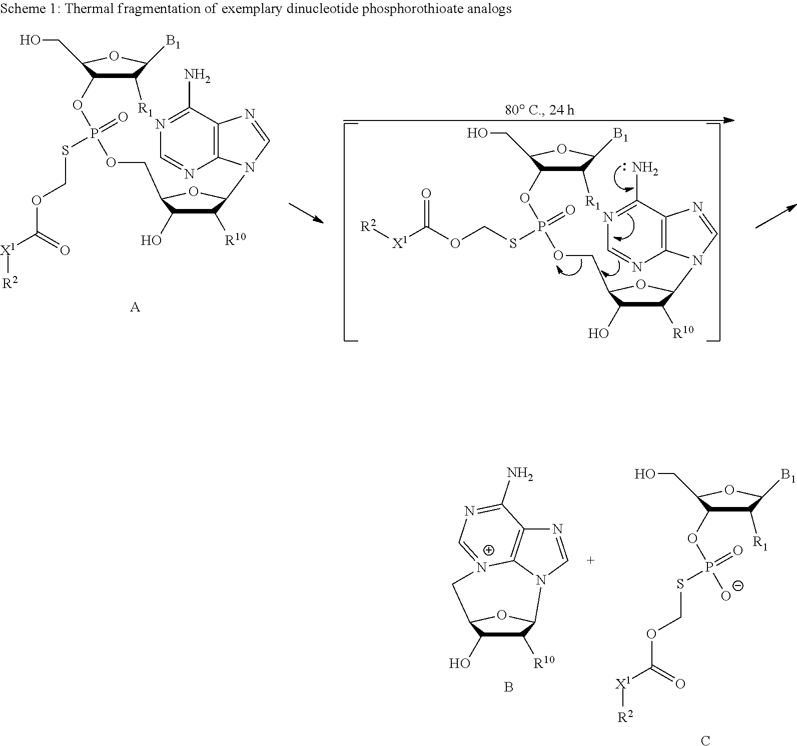

TABLE 1
Thermal fragmentation of exemplary dinucleotide phosphorothioate analogs

| Compound | | | | | | | |
|---|---|---|---|---|---|---|---|
| No. | R² | X¹ | B¹ | B² | R¹ | R¹⁰ | % Degrad. |
| 1 RpSp | t-Bu | — | T | A | H | H | 78% |
| 2 Isomer 1 | | | | | | | 30% |
| 3 Isomer 2 | | | | | | | 21% |
| 4 | t-Bu | — | T | T | H | H | ND |
| 5 | t-Bu | | T | C | H | H | 58% |
| 6 | t-Bu | — | U | A | OCH₃ | H | 28% |
| 7 | Boc—NH—X—CH₂—Ph | CH | U | A | OCH₃ | H | 58% |
| 8 | AcNH—X—CH₂—Ph | CH | U | A | OCH₃ | H | 72% |
| 9 RpSp | i-Pr | O | T | A | H | H | 30% |
| 10 Isomer 1 | | | | | | | 45% |
| 11 Isomer 2 | | | | | | | 42% |
| 12 | i-Pr | O | T | T | H | H | ND |
| 13 | i-Pr | O | T | C | H | H | 58% |
| 14 RpSp | i-Pr | O | U | A | OCH₃ | H | 20% |
| 15 Isomer 1 | | | | | | | ND |
| 16 Isomer 2 | | | | | | | 39% |
| 17 | i-Pr | O | U | A | OCH₃ | OCH₃ | 90% |
| 18 | i-Pr | O | U^{C}₅^{H}₈ | A | H | H | 42% |
| 19 | i-Pr | O | U^{C}₅^{H}₈ | A | OCH₃ | H | 53% |
| 20 | t-Bu—O—CH₂CH₂ | O | U | A | OCH₃ | H | 100% |
| 21 | i-Pr | O | U | A^{NHCOO—iPr} | OCH₃ | H | 67% |
| 22 | i-Pr | O | U | A^{bz2} | OCH₃ | H | 26% |

The fragmentation products were identified as the cyclonucleosides (B) and phosphorothioate diesters (C) shown in Scheme 1. Analysis of the experimental results suggests that the rate of the reaction is influenced by the nature of the S-alkyl groups, substitutions on the sugar rings and the nucleosidic bases. Furthermore, p-chirality had significantly more impact on the rate of fragmentation of the Sₚ-diastereomer, which was faster compared to that of the Rₚ-diastereomer.

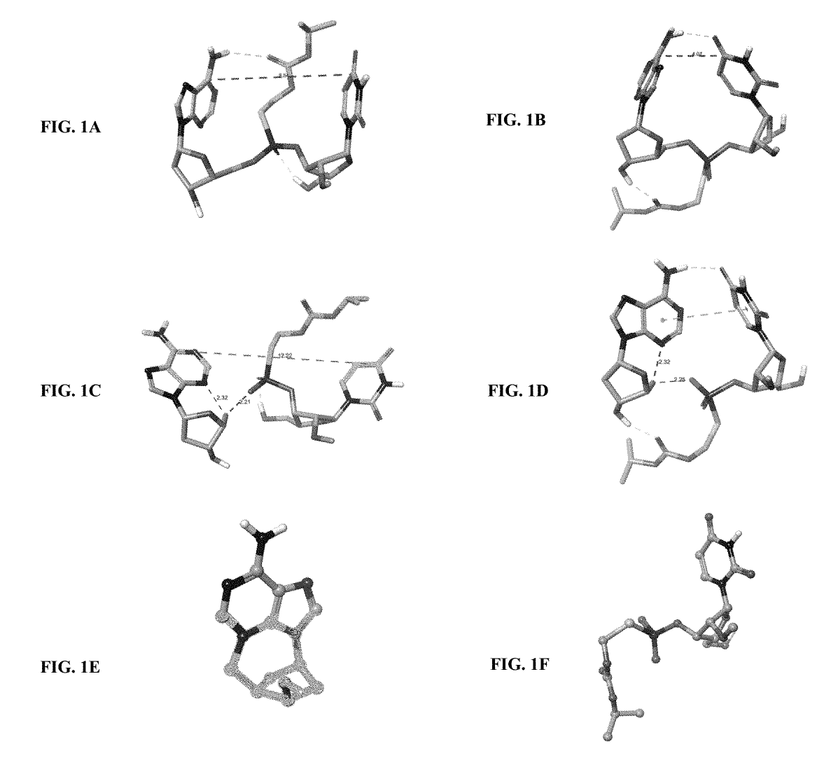

Quantum mechanical studies performed at low energy states optimized at the B3LYP 6-31G level revealed the likely conformations of each isomer and are shown in FIGS. 1A-1F. Without being bound by theory, in the case of the dinucleotide phosphotriesters, the S-alkyl side chain attached to the P chiral center may dictate the energetically favored conformations in the ground state (local) and transition state. In Rₚ-isomers, the S-alkyl side chain blocks the nucleobase interactions including hydrogen bonding interactions between purine and pyrimidine that is present in the Sₚ-isomer. In the case of the Sₚ-isomer, this conformation state is also “locked” in the transition state through additional pi-bond interactions between the nucleobases. Consequently, the Sₚ-dinucleotide is favorably poised towards cleavage of the (sugar-CH₂)—OP and formation of the cyclonucleoside B. Indeed, absence of the hydrogen bonding interactions between the nucleobases favors the “Rₚ-isomer like” conformation, which may offer higher protection against thermal fragmentation of the dinucleotide.

### EQUIVALENTS

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

What is claimed is:

1. A compound of Formula (I): 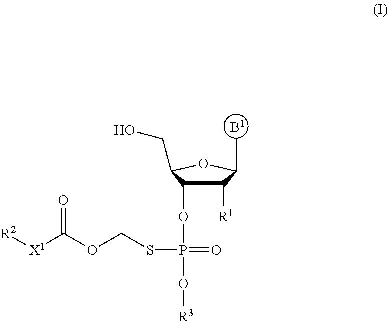
or a pharmaceutically acceptable salt thereof, wherein:
B¹ is adeninyl, cytosinyl, guanosinyl, thyminyl, uracilyl, or a modified nucleobase;
X¹ is absent, O, or C₁₋₆ alkyl;
R¹ is OH or O—(C₁₋₆ alkyl);
R² is C₁₋₆ alkyl, C₁₋₆ heteroalkyl, or arylalkyl, wherein each alkyl, heteroalkyl, or arylalkyl is optionally substituted with 1-5 R⁴;
R³ is absent or H;
R⁴ is C₁₋₆ alkyl, C₁₋₆ heteroalkyl, aryl, C(O)R^{a}, or C(O)OR^{b}; and
each of R^{a} and R^{b} is independently H, C₁₋₆ alkyl, or aryl.

2. The compound of claim 1, wherein B¹ is thyminyl, uracilyl, or a modified nucleobase.

3. The compound of claim 2, wherein B¹ is uracilyl or a substituted uracilyl.

4. The compound of claim 1, wherein X¹ is absent, O, or CH₂.

5. The compound of claim 1, wherein R¹ is OH or OCH₃.

6. The compound of claim 1, wherein R² is C₁₋₆ alkyl (e.g., C(CH₃)₃, CH(CH₃)₂).

7. The compound of claim 6, wherein R² is C₁₋₆ heteroalkyl (e.g., CH(NH(R⁴))CH₂R⁴, CH₂CH₂OC(CH₃)₃).

8. The compound of claim 1, wherein R⁴ is aryl (e.g., phenyl), C(O)R^{a} (e.g., C(O)CH₃) or C(O)OR^{b} (e.g., C(O)OC(CH₃)₃).

9. The compound of claim 1, wherein R³ is absent.

10. The compound of claim 1, wherein the compound of Formula (I) is a compound of Formula (I-a): 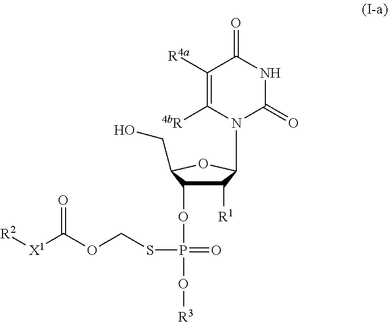
or a pharmaceutically acceptable salt thereof, wherein each of R¹, R², and R³ are as defined in claim 1, and each of R^{4a} and R^{4b} is independently H or C₁₋₆ alkyl, C₁₋₆ alkenyl, or C₁₋₆ alkynyl.

11. The compound of claim 10, wherein each of R^{4a} and R^{4b} is independently H.

12. The compound of claim 10, wherein one of R^{4a} and R^{4b} is independently H and the other of R^{4a} and R^{4b} is C₁₋₆ alkyl, C₁₋₆ alkenyl, or C₁₋₆ alkynyl.

13. The compound of claim 10, wherein one of R^{4a} and R^{4b} is independently H and the other of R^{4a} and R^{4b} is C₁₋₆ alkynyl (e.g., pentynyl).

14. A compound of Formula (II): 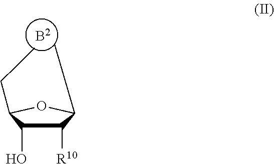
or a pharmaceutically acceptable salt thereof, wherein:
B² is adeninyl, cytosinyl, guanosinyl, thyminyl, uracilyl, or a modified nucleobase; and
R¹⁰ is OH or O—(C₁₋₆ alkyl).

15. The compound of claim 14, wherein B² is adeninyl, cytosinyl, thyminyl, or a modified nucleobase.

16. The compound of claim 15, wherein B² is adeninyl or a substituted adeninyl.

17. The compound of claim 14, wherein R¹⁰ is OH or OCH₃.

18. The compound of claim 14, wherein the compound of Formula (II) is a compound of Formula (II-a): 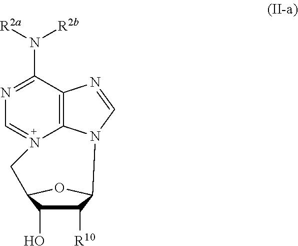
or a pharmaceutically acceptable salt thereof, wherein
R¹⁰ is OH or O—(C₁₋₆ alkyl);
each of R^{2a} and R^{2b} is independently H, C₁₋₆ alkyl, halogen, C(O)R^{c}, or C(O)OR^{d};
and each of R^{c} and R^{d} is independently H, C₁₋₆ alkyl, or aryl.

19. The compound of claim 18, wherein each of R^{2a} and R^{2b} is independently H or C(O)OR^{b}(e.g., C(O)OCH(CH₃)₂).

20. The compound of claim 18, wherein each of R^{2a} and R^{2b} is independently H.

21. The compound of claim 18, wherein one of R^{2a} and R^{2b} is H and the other of R^{2a} and R^{2b} is C(O)R^{c} or C(O)OR^{d} (e.g., C(O)-aryl or C(O)O—C₁₋₆ alkyl).

22. The compound of claim 18, wherein one of R^{2a} and R^{2b} is H and the other of R^{2a} and R^{2b} is C(O)R^{c} or C(O)OR^{d} (e.g., C(O)-phenyl or C(O)O—CH(CH₃)₂).

23. The compound of claim 14, wherein the compound of Formula (II) is a compound of Formula (II-b): 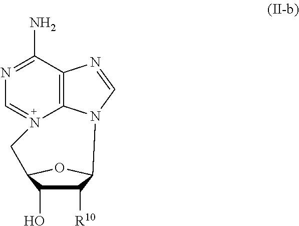
or a pharmaceutically acceptable salt thereof, wherein R¹⁰ is OH or O—(C₁₋₆ alkyl).

24. The compound of claim 1, wherein the compound is a byproduct of degradation.

25. The compound of claim 1, wherein the degradation is chemical degradation, physical degradation, or pH-related degradation.

26. The compound of claim 25, wherein the degradation is thermal degradation.

27. A pharmaceutical composition comprising a compound of Formula (III): 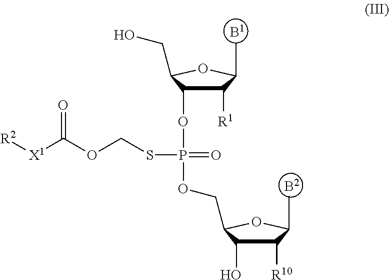
or a pharmaceutically acceptable salt thereof, wherein:
each of B¹ and B² is adeninyl, cytosinyl, guanosinyl, thyminyl, uracilyl, or a modified nucleobase;
X¹ is absent, O, or C₁₋₆ alkyl;
R¹ is OH or O—(C₁₋₆ alkyl);
R² is C₁₋₆ alkyl, C₁₋₆ heteroalkyl, or arylalkyl, wherein each alkyl, heteroalkyl, or arylalkyl is optionally substituted with 1-5 R⁴;
R⁴ is C₁₋₆ alkyl, C₁₋₆ heteroalkyl, aryl, C(O)R^{a}, or C(O)OR^{b};
R¹⁰ is H or O—(C₁₋₆ alkyl);
each of R^{a} and R^{b} is independently H, C₁₋₆ alkyl, or aryl;
and the composition is enantiomerically enriched for the Rp isomer over the Sp isomer.

28. The composition of claim 27, wherein each of B¹ and B² is independently adeninyl, thyminyl, uracilyl, or a modified nucleobase.

29. The composition of claim 27, wherein each of B¹ is independently thyminyl, uracilyl, or a modified nucleobase.

30. The composition of claim 29, wherein B¹ is uracilyl or a substituted uracilyl.

31. The composition of claim 27, wherein B² is adeninyl, cytosinyl, thyminyl, or a modified nucleobase.

32. The composition of claim 31, wherein B² is adeninyl or a substituted adeninyl.

33. The composition of claim 27, wherein X¹ is absent, O, or CH₂.

34. The composition of claim 27, wherein R¹ is OH or OCH₃.

35. The composition of claim 27, wherein R¹⁰ is OH or OCH₃.

36. The composition of claim 27, wherein R² is C₁₋₆ alkyl (e.g., C(CH₃)₃, CH(CH₃)₂).

37. The composition of claim 27, wherein R² is C₁₋₆ heteroalkyl (e.g., CH(NH(R⁴))CH₂R⁴, CH₂CH₂OC(CH₃)₃).

38. The composition of claim 27, wherein R⁴ is aryl (e.g., phenyl), C(O)R^{a} (e.g., C(O)CH₃) or C(O)OR^{b} (e.g., C(O)OC(CH₃)₃).

39. The composition of claim 27, wherein the composition comprises an ee ratio greater than about 51:49 of the Rp isomer to the Sp isomer.

40. The composition of claim 27, wherein the composition comprises an ee ratio greater than about 60:40 of the Rp isomer to the Sp isomer.

41. The composition of claim 27, which is substantially free of a compound of Formula (I): 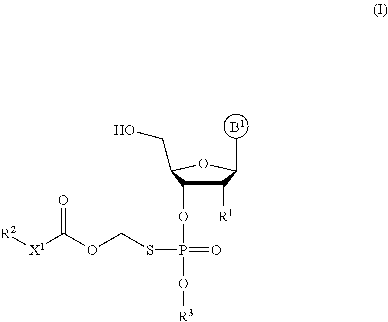
or a pharmaceutically acceptable salt thereof, wherein:
B¹ is adeninyl, cytosinyl, guanosinyl, thyminyl, uracilyl, or a modified nucleobase;
X¹ is absent, O, or C₁₋₆ alkyl;
R¹ is OH or O—(C₁₋₆ alkyl);
R² is C₁₋₆ alkyl, C₁₋₆ heteroalkyl, or arylalkyl, wherein each alkyl, heteroalkyl, or arylalkyl is optionally substituted with 1-5 R⁴;
R³ is absent or H;
R⁴ is C₁₋₆ alkyl, C₁₋₆ heteroalkyl, aryl, C(O)R^{a}, or C(O)OR^{b}; and
each of R^{a} and R^{b} is independently H, C₁₋₆ alkyl, or aryl;
or a compound of Formula (II): 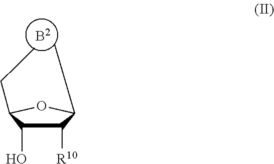
or a pharmaceutically acceptable salt thereof, wherein:
B² is adeninyl, cytosinyl, guanosinyl, thyminyl, uracilyl, or a modified nucleobase; and
R¹⁰ is OH or O—(C₁₋₆ alkyl);
each of R^{2a} and R^{2b} is independently H, C₁₋₆ alkyl, halogen, C(O)R^{c}, or C(O)OR^{d};
and each of R^{c} and R^{d} is independently H, C₁₋₆ alkyl, or aryl.

42. A dosage form comprising a pharmaceutical composition, wherein the composition comprises a compound of Formula (III): 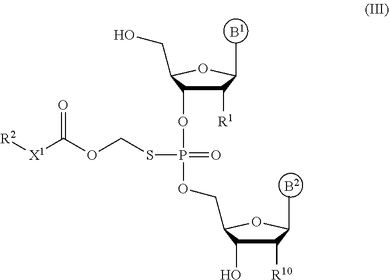
or a pharmaceutically acceptable salt thereof, wherein:
each of B¹ and B² is adeninyl, cytosinyl, guanosinyl, thyminyl, uracilyl, or a modified nucleobase;
X¹ is absent, O, or C₁₋₆ alkyl;
R¹ is OH or O—(C₁₋₆ alkyl);
R² is C₁₋₆ alkyl, C₁₋₆ heteroalkyl, or arylalkyl, wherein each alkyl, heteroalkyl, or arylalkyl is optionally substituted with 1-5 R⁴;
R⁴ is C₁₋₆ alkyl, C₁₋₆ heteroalkyl, aryl, C(O)R^{a}, or C(O)OR^{b};
R¹⁰ is H or O—(C₁₋₆ alkyl);
each of R^{a} and R^{b} is independently H, C₁₋₆ alkyl, or aryl;
and the composition is substantially free of a compound of Formula (I): 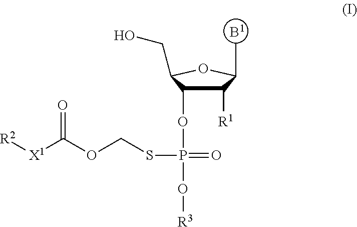
or a pharmaceutically acceptable salt thereof, wherein:
B¹ is adeninyl, cytosinyl, guanosinyl, thyminyl, uracilyl, or a modified nucleobase;
X¹ is absent, O, or C₁₋₆ alkyl;
R¹ is OH or O—(C₁₋₆ alkyl);
R² is C₁₋₆ alkyl, C₁₋₆ heteroalkyl, or arylalkyl, wherein each alkyl, heteroalkyl, or arylalkyl is optionally substituted with 1-5 R⁴;
R³ is absent or H;
R⁴ is C₁₋₆ alkyl, C₁₋₆ heteroalkyl, aryl, C(O)R^{a}, or C(O)OR^{b}; and
each of R^{a} and R^{b} is independently H, C₁₋₆ alkyl, or aryl.
or a compound of Formula (II): 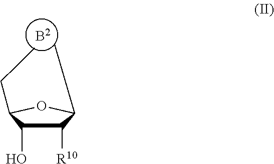
or a pharmaceutically acceptable salt thereof, wherein:
B² is adeninyl, cytosinyl, guanosinyl, thyminyl, uracilyl, or a modified nucleobase; and
R¹⁰ is OH or O—(C₁₋₆ alkyl);
each of R^{2a} and R^{2b} is independently H, C₁₋₆ alkyl, halogen, C(O)R^{c}, or C(O)OR^{d};
and each of R^{c} and R^{d} is independently H, C₁₋₆ alkyl, or aryl.

43. The dosage form of claim 42, wherein the dosage form is administered orally, parenterally, or topically.

44. The dosage form of claim 42, wherein the dosage form is administered through inhalation.

45. The dosage form of claim 42, wherein the dosage form comprises a solid or liquid.

46. The dosage form of claim 45, wherein the liquid dosage form comprises a suspension, a solution, a linctus, an emulsion, a drink, an elixir, or a syrup.

47. The dosage form of claim 45, wherein the solid dosage form comprises a capsule, tablet, dragée, or powder.

48. The dosage form of claim 42, wherein the composition further comprises an additive or preservative (e.g., PEG 400 or glycerin).

49. The dosage form of claim 42, wherein the composition further comprises an excipient (e.g., methylcellulose, talc, lactose, or starch).
